(19) United States
(12) Patent Application Publication
(10) Pub. No.: US 2020/0121448 A1
(43) Pub. Date: Apr. 23, 2020

(54) EXTENDED DEPTH OF FOCUS INTRAOCULAR LENS

(71) Applicant: ALCON INC., Fribourg (CH)

(72) Inventors: MYOUNG-TAEK CHOI, ARLINGTON, TX (US); GANG HUANG, GRAPEVINE, TX (US); YUEAI LIU, ARLINGTON, TX (US); MICHAEL LEE MANGUM, SUNNYVALE, CA (US); ZEYU ZHAO, SUNNYVALE, CA (US)

(21) Appl. No.: 16/655,481

(22) Filed: Oct. 17, 2019

Related U.S. Application Data
(60) Provisional application No. 62/747,578, filed on Oct. 18, 2018.

(51) Int. Cl. A61F 2/16

(52) U.S. Cl. CPC A61F 2/1618; A61F 2230/0023; A61F 2230/0021

(57) ABSTRACT

An intraocular lens including an optic zone and a modulated surface profile formed in the optic zone and configured to focus incident light at a plurality of focal points, wherein the modulated surface profile is incorporated with a base surface profile of the optic zone.

### BACKGROUND

### Field of the Disclosure

The present disclosure relates to ophthalmic lenses, such as intraocular lenses (IOLs), and more specifically, to an extended depth of focus intraocular lens.

### Description of the Related Art

The human eye includes a cornea and a crystalline lens that are intended to focus light that enters the pupil of the eye onto the retina. However, the eye may exhibit various refractive errors which result in light not being properly focused upon the retina, and which may reduce visual acuity. Ocular aberrations can range from the relatively simple spherical and cylindrical errors that cause myopia, hyperopia, or regular astigmatism, to more complex refractive errors that can cause, for example, halos and starbursts in a person's vision.

Many interventions have been developed over the years to correct various ocular aberrations. These include spectacles, contact lenses, corneal refractive surgery, such as laser-assisted in situ keratomileusis (LASIK) or corneal implants, and intraocular lenses (IOLs). The diagnosis and specification of sphero-cylindrical spectacles and contact lenses for treatment of myopia, hyperopia, and astigmatism are also well-established.

During cataract surgery or human natural lens replacement, an intraocular lens (IOL) is typically implanted in a patient's eye to compensate for the lost optical power when the natural lens is removed. The optimal outcome of cataract surgery is for the surgeon to achieve emmetropia such that the patient experiences 20/20 vision following the procedure and additional interventions are not needed. One of the determining factors for achieving emmetropia is precise placement of the lens inside the eye. Other factors for achieving emmetropia are pre-operative measurements, surgical technique, IOL design, and surgical experience. Current IOL designs require a surgeon to place an IOL within an approximately 0.1 mm window in the eye, i.e., an error allowance of ±0.05 mm. A patient's vision may be negatively impacted by modest post-surgery residual refractive errors in the treated eye(s).

Accordingly, there is a need for a system that provides an extended depth of focus IOL to reduce the impact that variations in lens placement and surgical technique have on the outcome of procedures.

### SUMMARY

The present disclosure provides an intraocular lens. The intraocular lens includes an optic zone, a modulated surface profile formed in the optic zone and configured to focus incident light at a plurality of focal points, wherein the modulated surface profile is incorporated with a base surface profile of the optic zone.

In additional embodiments, which may be combined with one another unless clearly exclusive: the intraocular lens wherein the plurality of focal points produce a through-focus modulation transfer function that is symmetric about a distance focal point such that at least one of the plurality of focal points is located myopic to the distance focal point and at least one of the plurality of focal points is located hyperopic to the distance focal point; the intraocular lens wherein the plurality of focal points includes a maximum myopic focal point and a maximum hyperopic focal point, and the maximum myopic focal point and the maximum hyperopic focal point are each within a range of 0.75 to 1.5 diopters from the distance focal point; the intraocular lens wherein each of the plurality of focal points has one or more corresponding nearest focal points, and each of the plurality of focal points is separated from the one or more corresponding nearest focal points by no more than 1 diopter; the intraocular lens wherein the modulated surface profile is a modified sinusoidal profile; the intraocular lens wherein the modified sinusoidal profile is a function of a radial position with respect to a center of the intraocular lens, and the modified sinusoidal profile is defined by a set of parameters including an amplitude parameter, a period parameter, and a phase constant parameter; the intraocular lens wherein the amplitude parameter and the period parameter are functions of the radial position; the intraocular lens wherein the modulated surface profile is a triangular profile; the intraocular lens wherein the triangular profile is a function of a radial position with respect to a center of the intraocular lens, the triangular profile includes a plurality of triangular peaks and a plurality of gaps, each of the peaks has an amplitude and a width, and each of the gaps has a width; the intraocular lens wherein the amplitude is the same for each of the plurality of peaks, the width of each of the plurality of peaks decreases as the radial position increases, and the width of each of the plurality of gaps decreases as the radial position increases; the intraocular lens wherein the triangular profile includes a flat portion at a center portion of the intraocular lens; the intraocular lens wherein the modulated surface profile is a square of sinusoidal profile; the intraocular lens wherein the square of sinusoidal profile is a function of a radial position with respect to a center of the intraocular lens, the square of sinusoidal profile is defined by a set of parameters including an amplitude parameter, a period parameter, and a phase constant parameter, and the square of sinusoidal profile includes a sign function component.

The present disclosure further provides an intraocular lens. The intraocular lens includes an optic zone, a plurality of surface regions of the optic zone, each of the plurality of surface regions having a dioptric power corresponding to a focal distance, the plurality of surface regions including a first surface region and a second surface region, the first surface region having a first dioptric power corresponding to a first focal distance, the first dioptric power further corresponding to a through-focus modulation transfer function having a peak performance and a focal shift corresponding to a percentage of the peak performance, the second surface region having a second dioptric power corresponding to a second focal distance, the second focal distance being offset from the first focal distance by at least the focal shift, and each of the plurality of surface regions having an area and configured to split incident light between the plurality of surface regions.

In additional embodiments, which may be combined with one another unless clearly exclusive: the intraocular lens wherein the first surface region further having a first radius and a first area, the second surface region extending from the first surface region to a second radius corresponding to a photopic aperture of a pupil, and the second surface region having a second area that is equal to the first area; the intraocular lens wherein the plurality of surface regions further includes a third surface region, the first surface region having a first radius and a first area, the second surface region extending from the first surface region to a second radius, the second surface region having a second area that is equal to the first area, the third surface region extending from the second surface region to a third radius corresponding to a mesopic aperture of a pupil, the third surface region having a third area that is equal to the second area, and the third surface region having a third dioptric power corresponding to a third focal distance; the intraocular lens wherein the focal shift corresponds to between 45 and 75 percent of the peak performance, and the second focal distance is offset from the first focal distance by between 1.5 and 2.5 times the focal shift; the intraocular lens wherein the focal shift corresponds to 50 percent of the peak performance, and the second focal distance is offset from the first focal distance by twice the focal shift; the intraocular lens wherein the second focal distance is offset from the first focal distance in a myopic direction; the intraocular lens wherein the second focal distance is offset from the first focal distance in a myopic direction, and the third focal distance is offset from the first focal distance by at least the focal shift in a hyperopic direction.

Any system described herein may be used with any method described herein and vice versa. It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

For a more complete understanding of the present invention and its features and advantages, reference is now made to the following description, taken in conjunction with the accompanying drawings, in which:

FIG. 1 is a depiction of an exemplary IOL;

FIG. 2 is a depiction of an example embodiment of an IOL with a plurality of surface regions;

FIG. 3 is a schematic of the example IOL shown in FIG. 2 focusing incident light at a plurality of focal points;

FIG. 4 shows a plot of the modulation transfer function in a human eye corresponding to the example IOL shown in FIG. 2 in comparison to the modulation transfer function corresponding to prior art IOLs;

FIG. 5 is a schematic of another example embodiment of an IOL focusing incident light at a plurality of oscillating focal points;

FIG. 6 shows a plot of an example embodiment of a modulated surface profile that may be used in the example IOL shown in FIG. 5;

FIG. 7 shows a plot of the resulting oscillating focal position as a function of the incident light position corresponding to the example modulated surface profile shown in FIG. 6;

FIG. 8 shows a plot of the resulting light intensity as a function of focal distance corresponding to the example modulated surface profile shown in FIG. 6;

FIG. 9 shows a plot of the modulation transfer function in a human eye corresponding to the example modulated surface profile shown in FIG. 6 in comparison to the modulation transfer function corresponding to a prior art IOL;

FIG. 10 shows a plot of the simulated visual acuity corresponding to the example modulated surface profile shown in FIG. 6 in comparison to the simulated visual acuity corresponding to a prior art IOL;

FIG. 11 shows a plot of another example embodiment of a modulated surface profile that may be used in the example IOL shown in FIG. 5;

FIG. 12 shows a plot of the resulting oscillating focal position as a function of the incident light position corresponding to the example modulated surface profile shown in FIG. 11;

FIG. 13 shows a plot of the modulation transfer function in a human eye corresponding to the example modulated surface profile shown in FIG. 11;

FIG. 14 shows a plot of another example embodiment of a modulated surface profile that may be used in the example IOL shown in FIG. 5;

FIG. 15 shows a plot of the resulting oscillating focal position as a function of the incident light position corresponding to the example modulated surface profile shown in FIG. 14; and

FIG. 16 shows a plot of the modulation transfer function in a human eye corresponding to the example modulated surface profile shown in FIG. 14.

### DESCRIPTION OF PARTICULAR EMBODIMENT(S)

The exemplary embodiments relate to ophthalmic devices such as IOLs and contact lenses. The following description is presented to enable one of ordinary skill in the art to make and use the invention and is provided in the context of a patent application and its requirements. Various modifications to the exemplary embodiments and the generic principles and features described herein will be readily apparent. The exemplary embodiments are mainly described in terms of particular methods and systems provided in particular implementations. However, the methods and systems will operate effectively in other implementations. For example, the method and system are described primarily in terms of IOLs. However, the method and system may be used with contact lenses and spectacle glasses.

In the following description, details are set forth by way of example to facilitate discussion of the disclosed subject matter. It should be apparent to a person of ordinary skill in the field, however, that the disclosed embodiments are exemplary and not exhaustive of all possible embodiments.

As used herein, a hyphenated form of a reference numeral refers to a specific instance of an element and the un-hyphenated form of the reference numeral refers to the collective element. Thus, for example, device ‘12-1’ refers to an instance of a device class, which may be referred to collectively as devices ‘12’ and any one of which may be referred to generically as a device ‘12’.

After cataract surgery, a patient will typically experience emmetropia or 20/20 vision in approximately 80% of procedures. As will be described in further detail, an extended depth of focus IOL is disclosed that, when used in a cataract procedure, will result in a greater number of procedures having the optimal outcome of emmetropia. Use of the extended depth of focus IOL may result in higher patient satisfaction, reduced probability of secondary surgical interventions such as explant, and a lower risk of visual acuity changes as the lens shifts or settles in the eye following the procedure. Patients treated with the extended depth of focus IOL may not require additional corrective spectacles, glasses, or contact lenses for distance vision after the cataract surgery. The extended depth of focus IOL may also be advantageously used in training for less-experienced surgeons as less perfect surgical technique and less sophisticated pre-operative measurements may be required to achieve emmetropia. Finally, the extended depth of focus IOL may allow for improved IOL designs and/or improved manufacturability of IOLs.

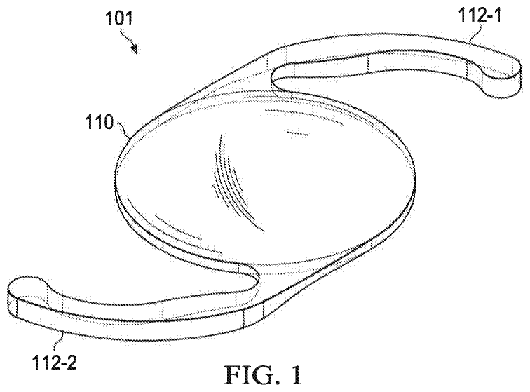

Referring now to the drawings, in FIG. 1, IOL 101 may represent any kind of IOL used in ophthalmology. As shown, IOL 101 includes an optic zone 110 (also referred to herein as simply an ‘optic’) and two haptics 112-1, 112-2, which are shown in an exemplary configuration for descriptive purposes. In various implementations, IOL 101 may include different types and numbers of haptics 112. In some implementations, IOL 101 may have no haptics. The materials used for optic zone 110 and haptics 112 may vary. For example, IOL 101 may be a non-foldable rigid IOL, such as with optic zone 110 comprising a polymethyl methacrylate (PMMA) lens. In some implementations, IOL 101 may be a flexible IOL, in which optic zone 110 may be comprised of various materials, such as silicone, hydrophobic acrylic, hydrophilic acrylic, hydrogel, collamer or combinations thereof. In IOL 101, haptics 112 may also be comprised of various materials, such as polypropylene, PMMA, hydrophobic acrylic, hydrophilic acrylic, silicone or combinations thereof. The optic zone 110 may be designed to have a specified optical refraction, or may be designed as a multi-focal element with a plurality of optical refraction powers. In particular, optic zone 110 may be implemented in an extended depth of focus IOL and may provide an extended range of vision around, for example, a distance focal point. Accordingly, the present disclosure is directed to modifications of the surface of a normal refractive monofocal IOL optic.

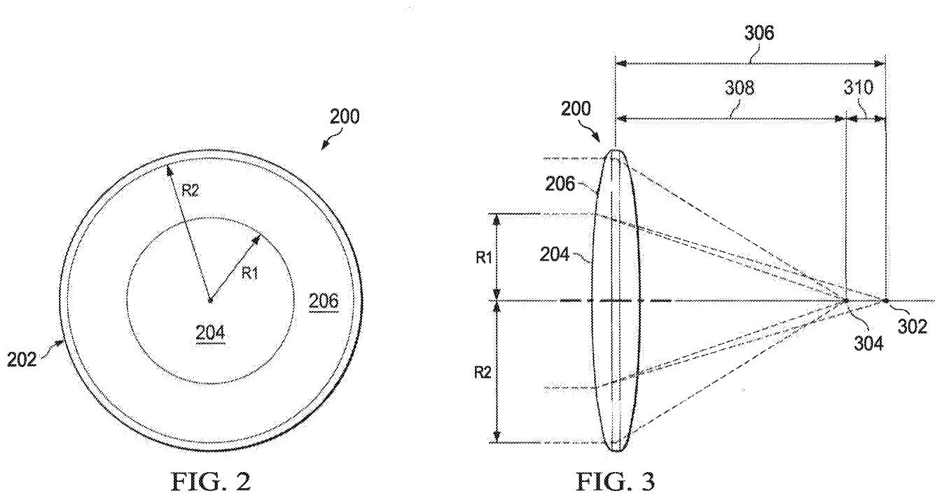

Referring now to FIG. 2, a depiction of an example embodiment of an IOL with a plurality of surface regions is shown. An IOL 200 may include an optic zone 202 that is divided into a plurality of surface regions, including first surface region 204 and second surface region 206. First surface region 204 and second surface region 206 may be concentric regions with their respective centers located at the center of optic zone 202. First surface region 204 may have a first area that may be defined as the area contained within a first radius R1. Second surface region 206 may have a second area that may be defined as the area contained between first radius R1 and a second radius R2.

In some instances, R2 may be defined to correspond to a photopic aperture of a human eye. A photopic aperture indicates a pupil aperture under well lit conditions, such as in daylight conditions or at ambient light intensities of about 3 candelas/square meter (cd/m²) or higher. A typical photopic aperture of a human eye is approximately 3 mm in diameter (or 1.5 mm radius). In other instances, R2 may be defined to correspond to a mesopic aperture of a human eye. A mesopic aperture is larger than the photopic aperture and indicates a pupil aperture under dimly lit conditions, such as under moonlight or at ambient light intensities between about 3 cd/m² and about 0.01 cd/m². A typical mesopic aperture of a human eye is approximately 5 mm in diameter (or 2.5 mm radius). In still other instances, R2 may be defined to correspond to some other size aperture diameter, e.g., 3.5 mm, 4 mm, 4.5 mm, or may be arbitrarily sized.

In some instances, R1 may be defined such that a first area of first surface region 204 is equal to a second area of second surface region 206. Defining R1 in this way results in approximately half of the incident light passing through first surface region 204 and half of the incident light passing through second surface region 206. Where first surface region 204 and second surface region 206 have equal areas, the following equation defines the relationship between R1 and R2:

$$R 1 = \frac{R 2 \sqrt{2}}{2}$$

For the case where R2 corresponds to a typical photopic aperture such that R2 is equal to 1.5 mm, the above equation results in R1 being equal to approximately 1.06 mm. Thus, where R2 is equal to 1.5 mm and R1 is equal to approximately 1.06 mm, first surface region 204 and second surface region 206 have approximately equal areas. R1 can similarly be calculated for any other value of R2.

In other instances, R1 may be defined such that the area of first surface region 204 is greater than or less than the area of second surface region 206. Selecting R1 may, therefore, allow for various designs of IOLs that split light between first surface region 204 and second surface region 206 in various proportions as needed for a given design.

Although FIG. 2 illustrates optic zone 202 with only two surface regions, other embodiments of IOLs may also be designed with optic zones having a larger number of surface regions. For example, an optic zone may be designed having three surface regions where the third surface region may have a third area defined as the area contained between a third radius R3 and second radius R2. Where the optic zone has three regions, R3 may be defined to correspond to a photopic aperture, a mesopic aperture, some other size aperture, or be arbitrarily sized. In some instances, R1 and R2 may be defined such that the areas of the first, second, and third surface regions are equal to each other. Defining R1 and R2 in this way results in approximately one third of the incident light passing through each of the surface regions. R1 and R2 can be calculated using similar principles as discussed above and based on a set value of R3. In other instances, R1 and R2 may be defined such that the surface regions have different areas with one or more of the surface regions having an area that is less than or greater than one or more of the other surface regions.

Referring now to FIG. 3, a schematic of the example IOL shown in FIG. 2 focusing incident light at a plurality of focal points is shown. As described above IOL 200 may include first surface region 204 and second surface region 206. First surface region 204 may be characterized by a first dioptric power such that incident light that passes through first surface region 204 is focused at a focal point 302. Second surface region 206 may be characterized by a second dioptric power such that incident light that passes through second surface region 206 is focused at a focal point 304. Focal point 302 is located a first focal distance 306 from IOL 200 and focal point 304 is located a second focal distance 308 from IOL 200. In general, a dioptric power may be related to a corresponding focal distance according to the following equation:

$$f = \frac{1000 mm}{φ}$$

where f is a focal distance and ϕ is a dioptric power. Therefore, by varying the first dioptric power selected for first surface region 204 and the second dioptric power selected for second surface region 206, the position of and separation between focal point 302 and focal point 304 may also be varied, and vice versa.

Focal point 302 is separated from focal point 304 by a distance 310. The position of focal point 302 and focal point 304 may be selected to achieve a through-focus modulation transfer function (MTF) in the approximate shape of a plateau throughout a focal range. For example, a desired value for distance 310 may be determined by identifying the defocus plane or focal shift at which the MTF of a monofocal lens reaches 50% of its maximum or peak performance. In other designs, a desired value for distance 310 may be determined by identifying the defocus plane or focal shift corresponding to a different percentage of the MTF peak performance, for example, between 45 and 75% of the MTF peak performance. In one example, the MTF of an SN60WF monofocal lens having 21.0D dioptric power may be simulated in a human model eye for a 3 mm pupil, at 35° C. with an image resolution of 100 lp/mm. In this example simulation, the lens achieves 50% of its MTF peak performance at a 0.065 mm focal shift in a human model eye. In this example, a plateau through focus MTF may be achieved by defining distance 310 as twice this focal shift or 0.13 mm. In other designs, distance 310 may be defined differently, for example, as at least the focal shift or as between 1.5 and 2.5 times the focal shift. Given that focal point 302 and focal point 304 are positioned closely, the MTF for each focal distance and dioptric power will most likely achieve 50% of the peak at approximately the same focal shift. Therefore, positioning focal point 302 and focal point 304 in this way results in overlapping MTF performance within the focal range associated with distance 310.

In this example, the first dioptric power of first surface region 204 is set to 21.0D and first focal distance 306 is calculated based on the equation above for a dioptric power of 21.0D. First back focal distance 306 in a human model eye may be 18.3 mm. Second focal distance 308 may then be offset by distance 310, which in this example is 0.13 mm or twice the focal shift. As shown in FIG. 3, focal point 304 is located myopic to focal point 302 such that second focal distance 308 is smaller in magnitude than first focal distance 306. However, in some designs of IOL 200, second focal distance 308 may be larger in magnitude than first focal distance 306 and focal point 304 may be located hyperopic to focal point 302. Second focal distance 308 may then be used to calculate the second dioptric power of second surface region 206. When focal point 304 is myopic to focal point 302 by twice the focal shift, the second dioptric power of second surface region 206 is set to 21.5 D. IOL 200, designed according to this example, may include first surface region 204 having a dioptric power of 21.0D and second surface region 206 having a dioptric power of 21.5 D.

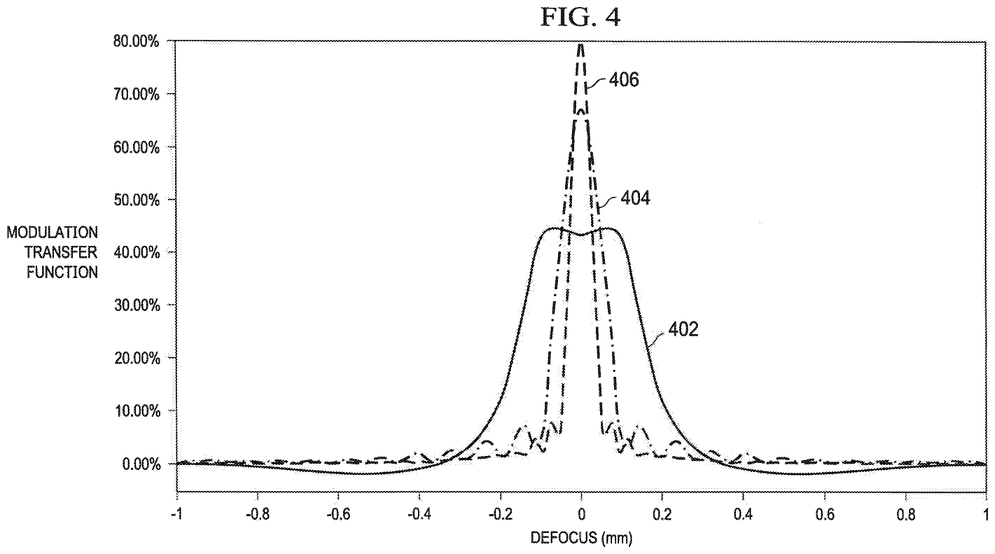

Referring now to FIG. 4, a plot of the modulation transfer function corresponding to the example IOL shown in FIG. 2 is shown in comparison to the modulation transfer function corresponding to prior art IOLs. Plot 402 shows the MTF performance of IOL 200 designed according to the example discussed above with respect to FIG. 3. Plot 404 shows the MTF performance of the SN60WF monofocal lens for a 3 mm photopic aperture condition, and plot 406 shows the MTF performance of the SN60WF monofolcal lens for a 5 mm mesopic aperture condition. As shown in FIG. 4, IOL 200 provides a plateau-like MTF performance for a broader range of focal distances than either of the monofocal lenses.

Although the above description in reference to FIGS. 3 and 4 describe the performance of a specific example of IOL 200, the scope of the disclosure is not so limited. For example, the first dioptric power of first surface region may be based on a different monofocal lens with a different dioptric power. Further, the simulation of the monofocal lens resulting in the MTF performance may be based on different inputs than described above, including, but not limited to, a different model eye, different temperature, image resolution, aperture conditions, etc. Finally, as discussed above in reference to FIG. 2, IOL 200 may have more than two surface regions. The principles described with respect to FIGS. 3 and 4 may be applied to an IOL with a larger number of surface regions. For example, an IOL may be designed with three surface regions where a second surface region and a third surface region are designed with a second dioptric power and a third dioptric power to focus incident light at focal points myopic and hyperopic, respectively, to a focal point associated with a first surface region. The focal distances of the myopic and hyperopic focal points may be offset from the first focal point by the same distance or by different distances. The offset distance may be at least the focal shift.

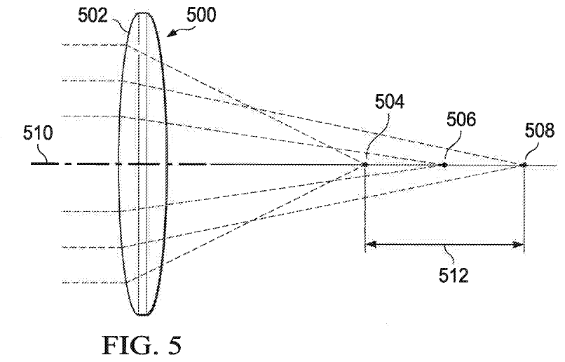

Referring now to FIG. 5, a schematic of another example embodiment of an IOL focusing incident light at a plurality of oscillating focal points is shown. An IOL 500 may include an optic zone (not expressly shown) that includes a modulated surface profile 502. Modulated surface profile 502 may be incorporated on one surface of a normal refractive monofocal IOL optic. Modulated surface profile 502 may be formed as a pattern within the same material as the base IOL optic itself. Modulated surface profile 502 may introduce a phase perturbation into an optical path of incident light resulting in two-sided extended depth of focus, for example, around a distance focus point. Incident light is focused at a plurality of alternating or oscillating focal points around a base focal point (not expressly shown), for example, focal points 504, 506, and 508.

As shown in FIG. 5, light is focused at different focal points depending on the incident light height or position with respect to an optical axis 510. For example, incident light near optical axis 510 may be focused at focal point 506, incident light near the periphery of IOL 500 may be focused at focal point 504, and incident light at an intermediate ray height may be focused at focal point 508. Although FIG. 5 illustrates only three focal points, the scope of the disclosure is not so limited. As will be described below in more detail, modulated surface profile 502 may be designed to focus light into a plurality of focal points or may be designed to focus light at continuous foci. For example, foci may be considered continuous when each of the plurality of focal points is no more than 1 diopter from each of its nearest focal points.

Incident light at different heights or positions relative to optical axis 510 may be focused onto different focal points due to local optical power variation of modulated surface profile 502 based on, for example, curvature and slope variation. Thus, IOL 500 may produce an extended depth of focus in a range 512. Range 512 may encompass focal points 504, 506, and 508 and may also encompass, for example, the distance focal point. At least one of the plurality of focal points may be myopic to, for example, the distance focal point, while at least one of the plurality of focal points may be hyperopic to the distance focal point. Range 512 may be defined by a maximum myopic focal point and a maximum hyperopic focal point. Range 512 may encompass approximately ±0.75 diopter to ±1.5 diopter with respect to, for example, the distance focal point. By alternating or oscillating the focal points at which incident light is focused, a symmetric extension of depth of focus may be achieved and may reduce the effects of both myopic and hyperopic refractive error. Alternating focal points also may decrease the pupil-size dependence such that a similar range of depth of focus extension occurs for both a photopic pupil condition and a mesopic pupil condition.

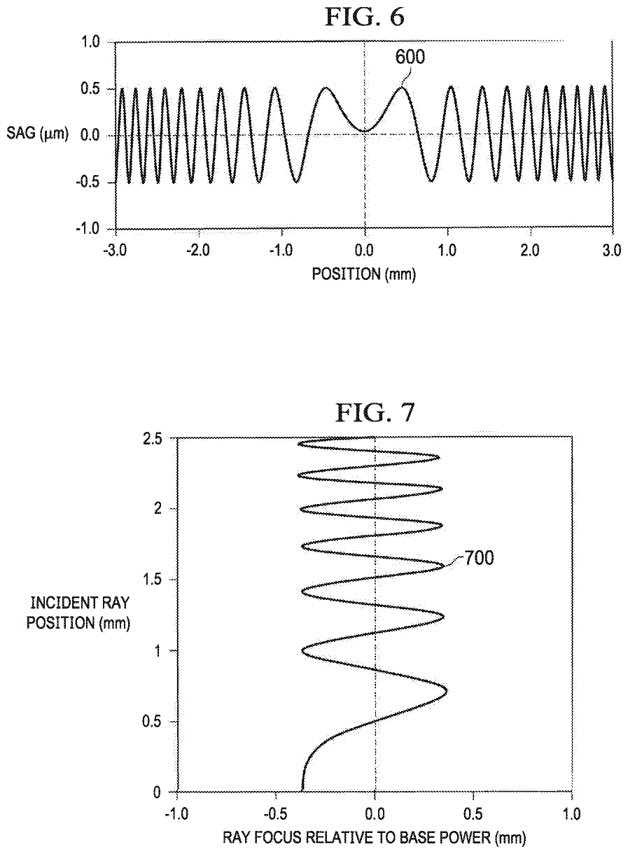

Referring now to FIG. 6, a plot of an example embodiment of a modulated surface profile that may be used in the example IOL shown in FIG. 5 is shown. A first example sag profile 600 may be used as modulated surface profile 502 shown in FIG. 5 above. Sag profile 600 may be a modified sinusoidal profile. In general, a surface profile of a lens, for example, IOL 500, may be represented as a sum of a base surface Z_{base} and a modulated surface profile Z_{MS} (Z=Z_{base}+Z_{MS}. The base surface Z_{base} may be defined by the following equation:

$${Z}_{base} = \frac{{cr}^{2}}{1 + \sqrt{1 - ( 1 + k ) {c}^{2} {r}^{2}}} + {A}_{4} {r}^{4} + {A}_{6} {r}^{6} + …$$

where c is the curvature, k is a conic constant, and A4 and A6 are aspheric coefficients.

Sag profile 600 may be defined by the following equation:

Z_{MS}=A(r)*sin(B(r)r²+Phi)

where A is the amplitude, B is associated with the period, and Phi is a phase constant of sag profile 600. A and B may both be functions of the radial position of incident light with respect to the center of the lens, r. A may be further defined as a polynomial expression of r:

A(r)=a+a1*r+a2*r²+a3*r³+ . . . +an*rⁿ

B may be further defined as a polynomial expression of r:

B(r)=b+b1*r+b2*r²+b3*r³+ . . . +an*rⁿ

For sag profile 600, the sinusoidal component may allow IOL 500 to generate continuous focus shifts. The phase constant Phi may allow IOL 500 to achieve a symmetric through focus MTF performance. In some instances, the amplitude A may include a position dependence, which may allow IOL 500 to have varying focus variation or pupil size dependence or apodization of the extension range. In other instances, the amplitude A may be constant such that sag profile 600 is the same for all pupil sizes. As shown in FIG. 6, sag profile 600 represents one example design of a sag profile where a=0.48 μm, b=0.458, Phi=4.8, and all other coefficients are set to zero. However, the scope of the disclosure is not so limited. For example, each of the coefficients and parameters of the above equations may be selected and adjusted to create a sag profile that results in the desired extended depth of focus for IOL 500.

Referring now to FIG. 7, a plot of the resulting oscillating focal position as a function of the incident light position corresponding to the example modulated surface profile shown in FIG. 6 is shown. Plot 700 illustrates how IOL 500 may focus incident light having various incident light positions when sag profile 600 is included in the optic zone. For example, as shown in FIG. 7, incident light passing through IOL 500 at a position approximately 1 mm from the center of the lens may be focused at a point approximately 0.4 mm myopic to a base focal distance, for example, the distance focal point. As shown in FIG. 7, sag profile 600 may result in a depth of focus extension of approximately ±0.4 mm relative to the base focal point. As discussed above in reference to FIG. 6, the parameters of sag profile 600 may be adjusted and doing so may also increase or decrease the depth of focus extension.

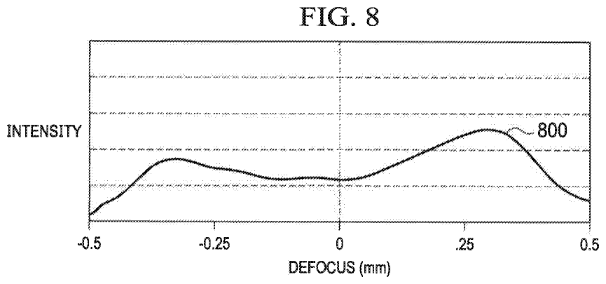

Referring now to FIG. 8, a plot of the resulting light intensity as a function of focal distance corresponding to the example modulated surface profile shown in FIG. 6 is shown. Plot 800 illustrates axial ray intensity at various focal distances and is generated using geometric ray tracing techniques. As shown in FIG. 8, plot 800 illustrates continuous distribution of rays around zero, which represents a base focal point, for example, the distance focal point. The ray intensity remains relatively high in a range of ±0.4 mm, which is similar to the depth of focus extension shown in FIG. 7.

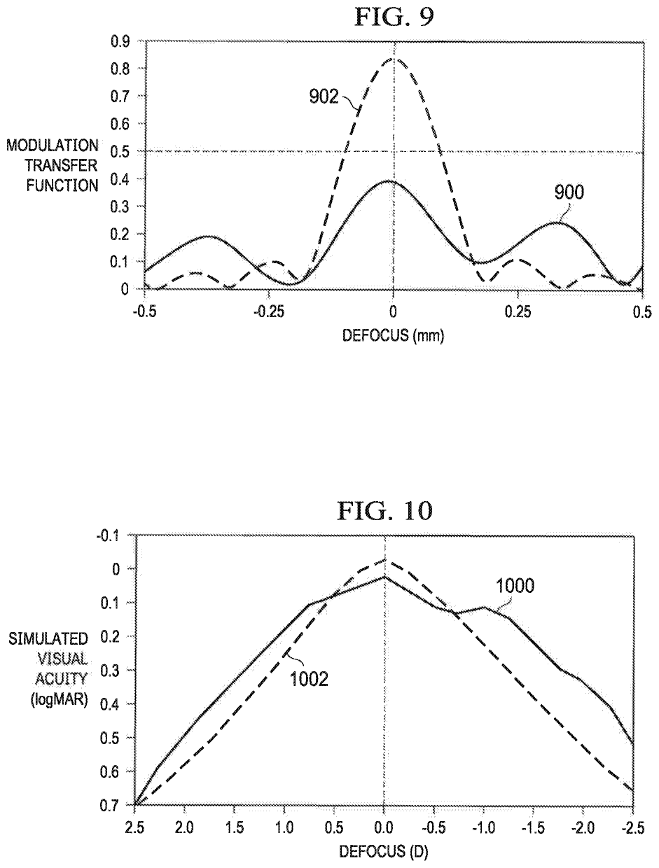

Referring now to FIG. 9, a plot of the modulation transfer function corresponding to the example modulated surface profile shown in FIG. 6 is shown in comparison to the modulation transfer function corresponding to a prior art IOL. Plot 900 represents the through focus MTF performance of IOL 500 when sag profile 600 is included in the optic zone. The spatial frequency of plot 900 is equivalent to a resolution of 20/40. For comparison, plot 902 represents the through focus MTF performance of a monofocal IOL. Plot 900 and plot 902 are generated by simulating the IOLs inside a human model eye. Plot 900 exhibits a similar depth of focus extension as shown in FIGS. 7 and 8. Plot 900 includes a peak at approximately 0.4 mm (or 1.0 diopter) on both the myopic and hyperopic side of a base focal distance, for example, the distance focal point. Plot 902 illustrates that the monofocal IOL has a MTF performance that approaches zero at these same positions.

Referring now to FIG. 10, a plot of the simulated visual quality corresponding to the example modulated surface profile shown in FIG. 6 is shown in comparison to the simulated visual acuity corresponding to a prior art IOL. Plot 1000 represents the vision quality of a model eye including IOL 500 when sag profile 600 is included in the optic zone. For comparison, plot 1002 represents the vision quality of a model eye including a monofocal IOL. Plot 1000 and plot 1002 are generated by simulating the model eye including the IOLs with a Monte-Carlo method using 200 virtual eyes incorporating clinical variation of biometric data. Plot 1000 illustrates that the visual acuity of IOL 500 with sag profile 600 can maintain a 0.1 LogMar performance, which is equivalent to 20/25 vision, in a range from +0.75 diopter to −1.0 diopter with modest post-surgery refractive errors. Plot 1002 illustrates that the visual acuity of the monofocal IOL may drop to 0.2 LogMar, which is equivalent to 20/32 vision, at these same positions.

Referring now to FIG. 11, a plot of another example embodiment of a modulated surface profile that may be used in the example IOL shown in FIG. 5 is shown. Another example sag profile 1100 may be used as modulated surface profile 502 shown in FIG. 5 above. Sag profile 1100 may be a triangular profile including a plurality of triangular peaks and a plurality of gaps between the peaks. Each of the peaks may have an amplitude and a width, while each of the gaps may have a width. Sag profile 1100 may be a function of a radial position with respect to the center of IOL 500. Further, each of the peaks may have the same amplitude or the amplitude may vary. The width of the peaks and gaps may also remain constant or vary. For example, the width of the peaks may decrease as radial position increases. The width of the gaps may also decrease as radial position increases. Sag profile 1100 may also include a flat portion 1102 at the center of IOL 500. Flat portion 1102 may send incident light to a distance focus, thereby improving the distance MTF performance. As shown in FIG. 11, sag profile 1100 represents one example design of a sag profile. However, the scope of the disclosure is not so limited. For example, various parameters, including but not limited to the presence or absence of a flat portion, width of the flat portion, peak amplitude, peak width, gap width, and number of peaks and gaps, of the sag profile may be selected and adjusted to create a sag profile that results in the desired extended depth of focus for IOL 500.

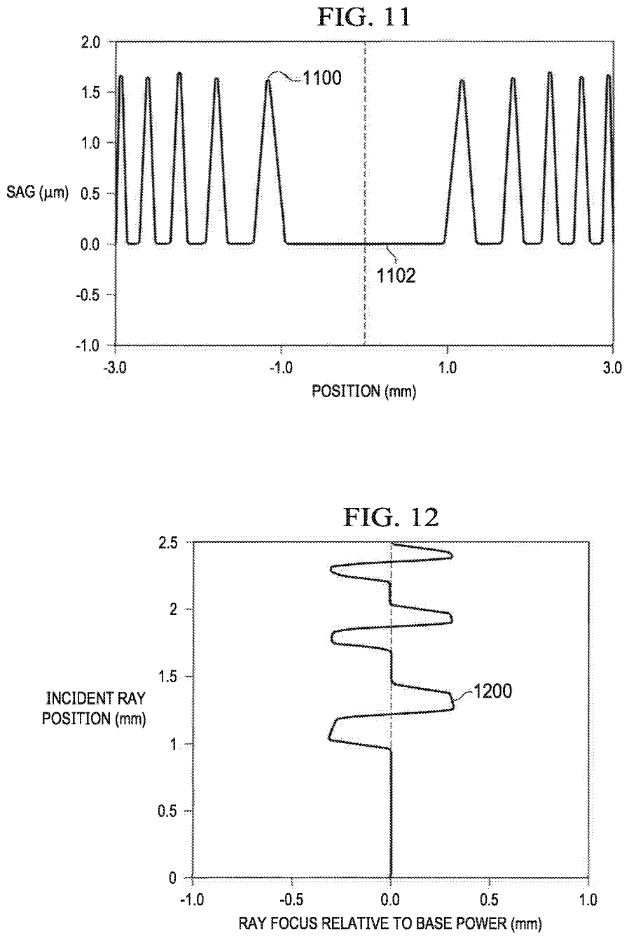

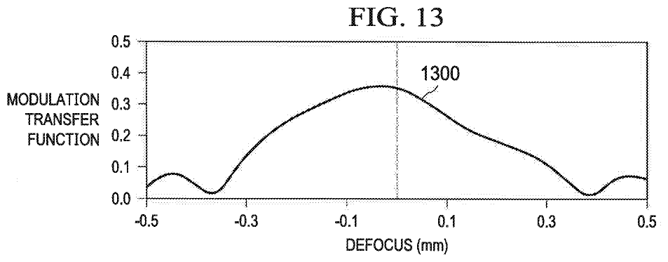

Referring now to FIG. 12, a plot of the resulting oscillating focal position as a function of the incident light position corresponding to the example modulated surface profile shown in FIG. 11 is shown. Plot 1200 illustrates how IOL 500 may focus incident light having various incident light positions when sag profile 1100 is included in the optic zone. For example, as shown in FIG. 12, incident light passing through IOL 500 at a position approximately 1 mm from the center of the lens may be focused at a point approximately 0.3 mm myopic to a base focal distance, for example, the distance focal point. As shown in FIG. 12, sag profile 1100 may result in a depth of focus extension of approximately ±0.3 mm relative to the base focal point. As discussed above in reference to FIG. 11, the parameters of sag profile 1100 may be adjusted and doing so may also increase or decrease the depth of focus extension.

Referring now to FIG. 13, a plot of the modulation transfer function corresponding to the example modulated surface profile shown in FIG. 11 is shown. Plot 1300 represents the through focus MTF performance of IOL 500 when sag profile 1100 is included in the optic zone. Plot 1300 is generated by simulating the IOL 500 with sag profile 1100 inside a human model eye. Plot 1300 exhibits a similar depth of focus extension as shown in FIG. 12. Plot 1300 shows that the MTF performance remains relatively high in a range of ±0.3 mm, which is similar to the depth of focus extension shown in FIG. 12.

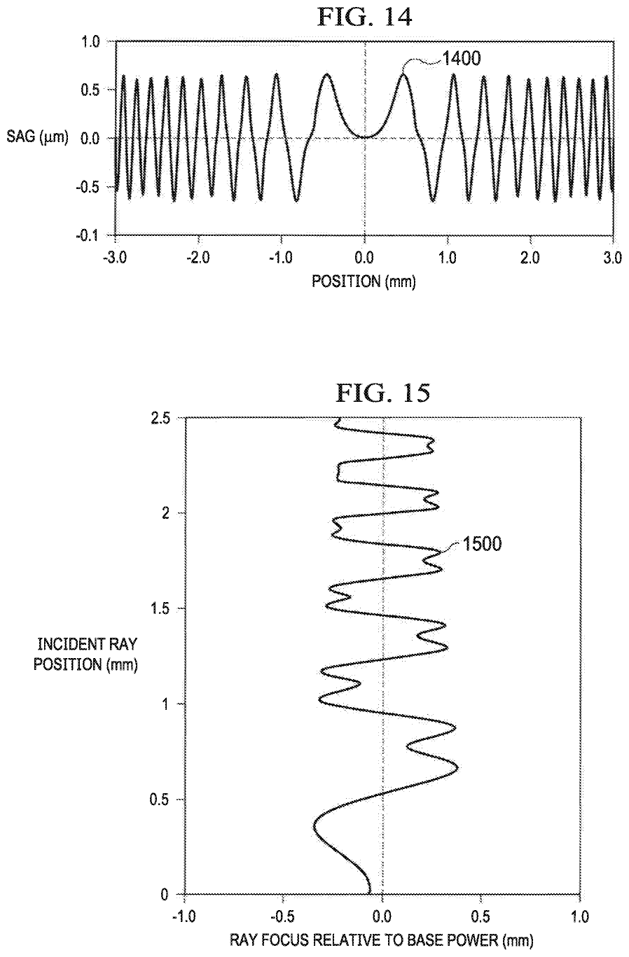

Referring now to FIG. 14, a plot of another example embodiment of a modulated surface profile that may be used in the example IOL shown in FIG. 5 is shown. Another example sag profile 1400 may be used as modulated surface profile 502 shown in FIG. 5 above. Sag profile 1400 may be a square of sinusoidal profile. Sag profile 1400 may be defined by the following equation:

Z_{MS}=Z1*Z1*sign(Z1)

where Z1 is further defined by the following equation:

Z1=A*cos(B*r*r+Phi)

where A is the amplitude, B is the period, Phi is a phase constant, and sign is a sign function. Sag profile 1400 may be a function of the radial position of incident light with respect to the center of the lens, r. As shown in FIG. 14, sag profile 1400 represents one example design of a sag profile where A=0.25 μm, B=6.85, Phi=4.808. However, the scope of the disclosure is not so limited. For example, each of the parameters of the above equations may be selected and adjusted to create a sag profile that results in the desired extended depth of focus for IOL 500.

Referring now to FIG. 15, a plot of the resulting oscillating focal position as a function of the incident light position corresponding to the example modulated surface profile shown in FIG. 14 is shown. Plot 1500 illustrates how IOL 500 may focus incident light having various incident light positions when sag profile 1400 is included in the optic zone. For example, as shown in FIG. 15, incident light passing through IOL 500 at a position approximately 1 mm from the center of the lens may be focused at a point approximately 0.3 mm myopic to a base focal distance, for example, the distance focal point. As shown in FIG. 15, sag profile 1400 may result in a depth of focus extension of approximately ±0.3 mm relative to the base focal point. As discussed above in reference to FIG. 14, the parameters of sag profile 1400 may be adjusted and doing so may also increase or decrease the depth of focus extension.

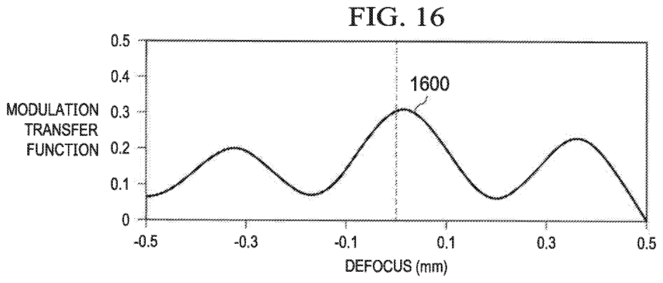

Referring now to FIG. 16, a plot of the modulation transfer function corresponding to the example modulated surface profile shown in FIG. 14 is shown. Plot 1600 represents the through focus MTF performance of IOL 500 when sag profile 1400 is included in the optic zone. Plot 1600 is generated by simulating the IOL 500 with sag profile 1400 inside a human model eye. Plot 1600 exhibits a similar depth of focus extension as shown in FIG. 15. Plot 1600 shows that the MTF performance remains relatively high in a range of ±0.3 mm, which is similar to the depth of focus extension shown in FIG. 15. Plot 1600 includes a peak at approximately 0.4 mm (or 1.0 diopter) on both the myopic and hyperopic side of a base focal distance, for example, the distance focal point.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. An intraocular lens comprising:
an optic zone;
a modulated surface profile formed in the optic zone and configured to focus incident light at a plurality of focal points,
wherein the modulated surface profile is incorporated with a base surface profile of the optic zone.

2. The intraocular lens of claim 1, wherein the plurality of focal points produce a through-focus modulation transfer function that is symmetric about a distance focal point such that at least one of the plurality of focal points is located myopic to the distance focal point and at least one of the plurality of focal points is located hyperopic to the distance focal point.

3. The intraocular lens of claim 2, wherein:
the plurality of focal points includes a maximum myopic focal point and a maximum hyperopic focal point; and
the maximum myopic focal point and the maximum hyperopic focal point are each within a range of 0.75 to 1.5 diopters from the distance focal point.

4. The intraocular lens of claim 1, wherein:
each of the plurality of focal points has one or more corresponding nearest focal points; and
each of the plurality of focal points is separated from the one or more corresponding nearest focal points by no more than 1 diopter.

5. The intraocular lens of claim 1, wherein the modulated surface profile is a modified sinusoidal profile.

6. The intraocular lens of claim 5, wherein:
the modified sinusoidal profile is a function of a radial position with respect to a center of the intraocular lens; and
the modified sinusoidal profile is defined by a set of parameters including an amplitude parameter, a period parameter, and a phase constant parameter.

7. The intraocular lens of claim 6, wherein the amplitude parameter and the period parameter are functions of the radial position.

8. The intraocular lens of claim 1, wherein the modulated surface profile is a triangular profile.

9. The intraocular lens of claim 8, wherein:
the triangular profile is a function of a radial position with respect to a center of the intraocular lens;
the triangular profile includes a plurality of triangular peaks and a plurality of gaps;
each of the peaks has an amplitude and a width; and
each of the gaps has a width.

10. The intraocular lens of claim 9, wherein:
the amplitude is the same for each of the plurality of peaks;
the width of each of the plurality of peaks decreases as the radial position increases; and
the width of each of the plurality of gaps decreases as the radial position increases.

11. The intraocular lens of claim 8, wherein the triangular profile includes a flat portion at a center portion of the intraocular lens.

12. The intraocular lens of claim 1, wherein the modulated surface profile is a square of sinusoidal profile.

13. The intraocular lens of claim 12, wherein:
the square of sinusoidal profile is a function of a radial position with respect to a center of the intraocular lens;
the square of sinusoidal profile is defined by a set of parameters including an amplitude parameter, a period parameter, and a phase constant parameter; and
the square of sinusoidal profile includes a sign function component.

14. An intraocular lens comprising:
an optic zone;
a plurality of surface regions of the optic zone;
each of the plurality of surface regions having a dioptric power corresponding to a focal distance;
the plurality of surface regions including a first surface region and a second surface region;
the first surface region having a first dioptric power corresponding to a first focal distance;
the first dioptric power further corresponding to a through-focus modulation transfer function having a peak performance and a focal shift corresponding to a percentage of the peak performance;
the second surface region having a second dioptric power corresponding to a second focal distance;
the second focal distance being offset from the first focal distance by at least the focal shift; and
each of the plurality of surface regions having an area and configured to split incident light between the plurality of surface regions.

15. The intraocular lens of claim 14, wherein:
the first surface region further having a first radius and a first area;
the second surface region extending from the first surface region to a second radius corresponding to a photopic aperture of a pupil; and
the second surface region having a second area that is equal to the first area.

16. The intraocular lens of claim 14, wherein:
the plurality of surface regions further includes a third surface region;
the first surface region having a first radius and a first area;
the second surface region extending from the first surface region to a second radius;
the second surface region having a second area that is equal to the first area;
the third surface region extending from the second surface region to a third radius corresponding to a mesopic aperture of a pupil;
the third surface region having a third area that is equal to the second area; and
the third surface region having a third dioptric power corresponding to a third focal distance.

17. The intraocular lens of claim 14, wherein:
the focal shift corresponds to between 45 and 75 percent of the peak performance; and
the second focal distance is offset from the first focal distance by between 1.5 and 2.5 times the focal shift.

18. The intraocular lens of claim 14, wherein:
the focal shift corresponds to 50 percent of the peak performance; and
the second focal distance is offset from the first focal distance by twice the focal shift.

19. The intraocular lens of claim 14, wherein the second focal distance is offset from the first focal distance in a myopic direction.

20. The intraocular lens of claim 16, wherein the second focal distance is offset from the first focal distance in a myopic direction; and
the third focal distance is offset from the first focal distance by at least the focal shift in a hyperopic direction.
